(19) United States
(12) Patent Application Publication
(10) Pub. No.: US 2023/0201403 A1
(43) Pub. Date: Jun. 29, 2023

(54) Emitter and System for Discharge of a Decontaminating Liquid-Gas Stream

(71) Applicant: VICTAULIC COMPANY, Easton, PA (US)

(72) Inventor: Rodrigo M. Feuchter, Chihuahua (MX)

(21) Appl. No.: 17/925,964

(22) PCT Filed: May 17, 2021
(86) PCT No.: PCT/IB2021/054221

§ 371 Date: Nov. 17, 2022

Related U.S. Application Data
(60) Provisional application No. 63/027,614, filed on May 20, 2020.

(51) Int. Cl. A61L 9/14; A61L 2/20; B05B 1/26; B05B 7/24

(52) U.S. Cl. CPC A61L 9/14; A61L 2/20; B05B 1/265; B05B 7/2489; A61L 2202/11

(57) ABSTRACT

A system for biological decontamination uses high velocity low pressure emitters to create a fog of liquid decontamination agent or liquid and gaseous decontamination agents to blanket a volume to be decontaminated. The liquid decontamination agent and atomizing gas are suppled under pressure to the emitters which create various shock fronts in the gas stream as the gas is discharged from the emitters. The liquid decontamination agent is entrained in the gas stream as it is also discharged from the emitter, and the shock fronts atomize the liquid decontamination agent into droplets which form the decontaminating fog.

### CROSS REFERENCE TO RELATED APPLICATIONS

This application is based upon and claims benefit of priority to U.S. Provisional Application No. 63/027,614, filed May 20, 2020, which application is hereby incorporated by reference herein.

### FIELD OF THE INVENTION

This invention concerns devices for emitting atomized decontamination agents, the device injecting the decontamination agent or agents into a gas flow stream where the agent or agents are atomized and projected away from the device.

### BACKGROUND

Biological decontamination equipment and methods according to the prior art are known to suffer various disadvantages. For example, manual techniques take considerable time, cannot be easily or practically applied in many situations and are effective in direct proportion to the care taken to apply the decontamination agent. Manual cleaning procedures, for example, have been documented to leave 30% to 60% of surface pathogens viable in hospital settings. Ultraviolet decontamination techniques suffer from shadowing effects, as there must be a direct line of sight from the light source to every surface to be decontaminated. Systems which use heat to vaporize decontamination agents require relatively high concentrations of active ingredients because the heat tends to disperse the vapor. Furthermore, prior art systems are known to be labor intensive, have unacceptably long cycle times (the time between application of decontamination agents and the next use of the decontaminated facility), many are not environmentally friendly and are not compatible with many other agents in use. There is clearly an opportunity to improve upon systems and methods for biological decontamination of various facilities.

### SUMMARY

One aspect of the invention concerns an emitter for atomizing and discharging a decontamination agent entrained in a gas stream. In an example embodiment the emitter is connectable in fluid communication with a pressurized source of the decontamination agent and a pressurized source of the gas. An example emitter embodiment comprises a nozzle having an inlet and an outlet and an unobstructed bore therebetween. The outlet has a diameter. The inlet is connectable in fluid communication with the pressurized gas source. A duct, separate from the nozzle is connectable in fluid communication with the pressurized source of the decontamination agent. The duct has an exit orifice separate from and positioned adjacent to the nozzle outlet. A deflector surface is positioned facing the nozzle outlet. The deflector surface is positioned in spaced relation to the nozzle outlet and has a first surface portion comprising a flat surface oriented substantially perpendicularly to the nozzle and a second surface portion comprising an angled or a curved surface surrounding the flat surface. The flat surface has a wetted area defined by a minimum diameter approximately equal to the outlet diameter. The decontamination agent is dischargeable from the orifice, and the gas is dischargeable from the nozzle outlet. The decontamination agent is entrained with the gas and atomized forming a decontamination agent liquid-gas stream that is deflected by the wetted area of the deflector surface and flows away therefrom.

The invention further encompasses a biological decontamination system. In an example embodiment the system comprises a source of pressurized gas, a source of pressurized decontamination agent, at least one emitter for atomizing and discharging the decontamination agent entrained in the gas, a gas conduit providing fluid communication between the pressurized gas source and the emitter, a piping network, separate from the gas conduit, the piping network providing fluid communication between the pressurized source of decontamination agent and the emitter, a first valve in the gas conduit controlling pressure and flow rate of the gas to the emitter, a second valve in the piping network controlling pressure and flow rate of the decontamination agent to the emitter, a pressure transducer measuring pressure within the gas conduit, and a control system in communication with the first and second valves and the pressure transducer, the control system receiving signals from the pressure transducer and opening and closing the valves in response to a signal indicative of the pressure within the gas conduit. In an example embodiment the emitter comprises a nozzle having an inlet and an outlet and an unobstructed bore therebetween. The inlet is connected in fluid communication with the first valve. The outlet has a diameter. A duct, separate from the nozzle, is connected in fluid communication with the second valve. The duct has an exit orifice separate from and positioned adjacent to the nozzle outlet. A deflector surface is positioned facing the nozzle outlet. The deflector surface is positioned in spaced relation to the nozzle outlet and has a first surface portion comprising a flat surface oriented substantially perpendicularly to the nozzle and a second surface portion comprising an angled or a curved surface surrounding the flat surface. The flat surface has a wetted area defined by a minimum diameter approximately equal to the outlet diameter.

The invention also covers a method of operating a biological decontamination system. In an example embodiment the system has an emitter comprising a nozzle having an unobstructed bore positioned between an inlet and an outlet. The nozzle inlet is connected in fluid communication with a pressurized gas source. The outlet has a diameter. A duct, separate from the nozzle, is connected in fluid communication with a pressurized source of decontamination agent. The duct has an exit orifice positioned adjacent to the nozzle outlet. A deflector surface is positioned facing the nozzle outlet in spaced relation thereto. The deflector surface comprises a flat surface oriented substantially perpendicularly to the nozzle. The flat surface has a wetted area defined by a minimum diameter approximately equal to the outlet diameter. In an example embodiment the method comprises:
discharging the decontamination agent from the exit orifice;
discharging the gas from the nozzle outlet, the gas achieving supersonic speed;
establishing a first shock front between the outlet and the deflector surface wherein the gas slows to subsonic speed and then impinges on the wetted area;
establishing a second shock front proximate to the deflector surface, the gas moving across the wetted area and increasing to supersonic speed between the first shock front and the second shock front, and decreasing in speed after passing through the second shock front;
entraining the decontamination agent in the gas proximate to the second shock front to form a decontamination liquid-gas stream; and projecting the decontamination liquid-gas stream from the emitter.

The invention also encompasses a method of operating a biological decontamination system. The example system has an emitter comprising a nozzle having an unobstructed bore positioned between an inlet and an outlet. The nozzle inlet is connected in fluid communication with a pressurized gas source. The outlet has a diameter. A duct, separate from the nozzle, is connected in fluid communication with a pressurized source of decontamination agent. The duct has an exit orifice positioned adjacent to the nozzle outlet. A deflector surface is positioned facing the nozzle outlet in spaced relation thereto. The deflector surface comprises a flat surface oriented substantially perpendicularly to the nozzle. The flat surface has a wetted area defined by a minimum diameter approximately equal to the outlet diameter. By way of example the method comprises:
discharging the decontamination agent from the exit orifice;
discharging the gas from the nozzle outlet, the gas achieving supersonic speed;
establishing a first shock front between the outlet and the deflector surface wherein the gas slows to subsonic speed and then impinges on the wetted surface;
establishing a second shock front proximate to the deflector surface, the gas moving across the wetted area and increasing to supersonic speed between the first shock front and the second shock front, and decreasing in speed after passing through the second shock front;
entraining the decontamination agent in the gas at at least one of the shock fronts to form a decontamination liquid-gas stream; and
projecting the decontamination liquid-gas stream from the emitter.

Further included in the invention is a biological decontamination system. In an example embodiment the system comprises a gaseous decontamination agent, a liquid decontamination agent, at least one emitter for atomizing and entraining the liquid decontamination agent in the gaseous decontamination agent and discharging the gaseous and liquid decontamination agents. A gas conduit conducts the gaseous decontamination agent to the emitter. A piping network conducts the liquid decontamination agent to the emitter. A first valve in the gas conduit controls pressure and flow rate of the gaseous decontamination agent to the emitter and a second valve in the piping network controls pressure and flow rate of the liquid decontamination agent to the emitter. A pressure transducer measures pressure within the gas conduit. In the example embodiment the emitter comprises a nozzle having an inlet and an outlet and an unobstructed bore therebetween. The outlet has a diameter. The inlet is connected with the gas conduit downstream of the first valve. A duct is connected in fluid communication with the piping network downstream of the second valve. The duct has an exit orifice positioned adjacent to the nozzle outlet. A deflector surface is positioned facing the nozzle outlet. The deflector surface is positioned in spaced relation to the nozzle outlet and has a first surface portion comprising a flat surface oriented substantially perpendicularly to a gas flow from the nozzle and a second surface portion comprising an angled or a curved surface surrounding the flat surface. The flat surface has a wetted area defined by a minimum diameter approximately equal to the outlet diameter. A control system is in communication with the first and second valves and the pressure transduce. The control system receives signals from the pressure transducer and opens and closes the valves in response thereto.

### BRIEF DESCRIPTION OF THE DRAWINGS

FIG. 1 is a longitudinal sectional view of a high velocity low pressure emitter according to the invention;

FIG. 2 is a longitudinal sectional view showing a component of the emitter depicted in FIG. 1;

FIG. 3 is a longitudinal sectional view showing a component of the emitter depicted in FIG. 1;

FIG. 4 is a longitudinal sectional view showing a component of the emitter depicted in FIG. 1;

FIG. 5 is a longitudinal sectional view showing a component of the emitter depicted in FIG. 1;

FIG. 6 is a diagram depicting fluid flow from the emitter based upon a Schlieren photograph of the emitter shown in FIG. 1 in operation;

FIG. 7 is a diagram depicting predicted fluid flow for another embodiment of the emitter;

FIG. 8 is a schematic diagram illustrating an example biological decontamination system according to the invention.

### DETAILED DESCRIPTION

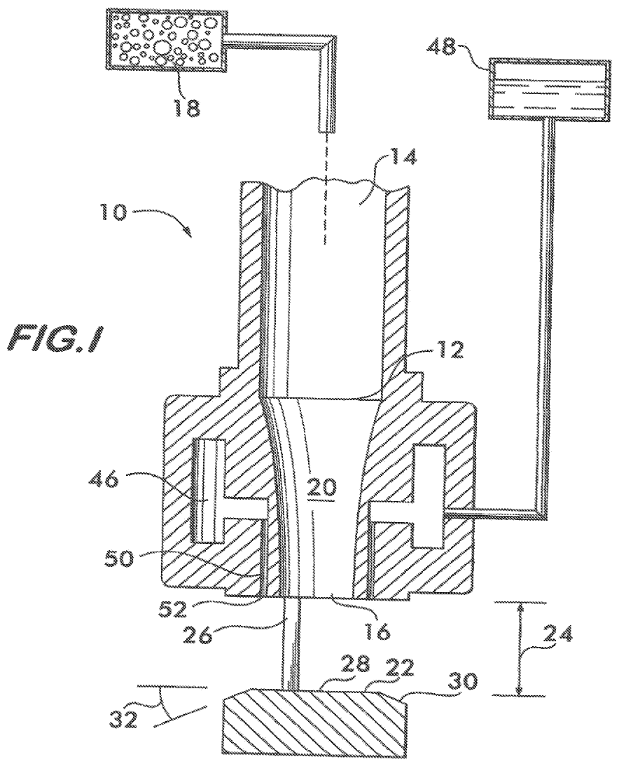

FIG. 1 shows a longitudinal sectional view of a high velocity low pressure emitter 10 for use with a biological decontamination system according to the invention. Emitter 10 comprises a convergent nozzle 12 having an inlet 14 an outlet 16 and an unobstructed bore therebetween. Outlet 16 may range in diameter from about ⅛ inch to about 1 inch for many applications. Inlet 14 is in fluid communication with a pressurized gas supply 18 that provides gas to the nozzle at a predetermined pressure and flow rate. Pressurized gas for use with a decontamination system may be, for example air, nitrogen, oxygen, argon-nitrogen mixtures, argon-carbon dioxide mixtures or may also comprise a biological decontamination agent such as ozone, halogens, halocarbons and ethylene oxide. It is advantageous that the nozzle 12 have a curved convergent inner surface 20, although other shapes, such as a linear tapered surface, are also feasible.

A deflector surface 22 is positioned facing and in spaced apart relation with the nozzle 12. A gap 24 is established between the deflector surface and the nozzle outlet. The gap may range in size from about 1/10 inch to about ¾ inches. The deflector surface 22 is held in spaced relation from the nozzle by one or more support legs 26.

Preferably, deflector surface 22 comprises a flat surface portion 28 substantially aligned with the nozzle outlet 16 and having a wetted area defined by a minimum diameter approximately equal to the nozzle outlet diameter. Deflector surface 22 further comprises an angled surface portion 30 contiguous with and surrounding the flat portion. Flat portion 28 is substantially perpendicular to the gas flow from nozzle 12, and has a minimum diameter approximately equal to the diameter of the outlet 16. The angled portion 30 is oriented at a sweep back angle 32 from the flat portion. The sweep back angle may range between about 15° and about 45° and, along with the size of gap 24, determines the dispersion pattern of the flow from the emitter.

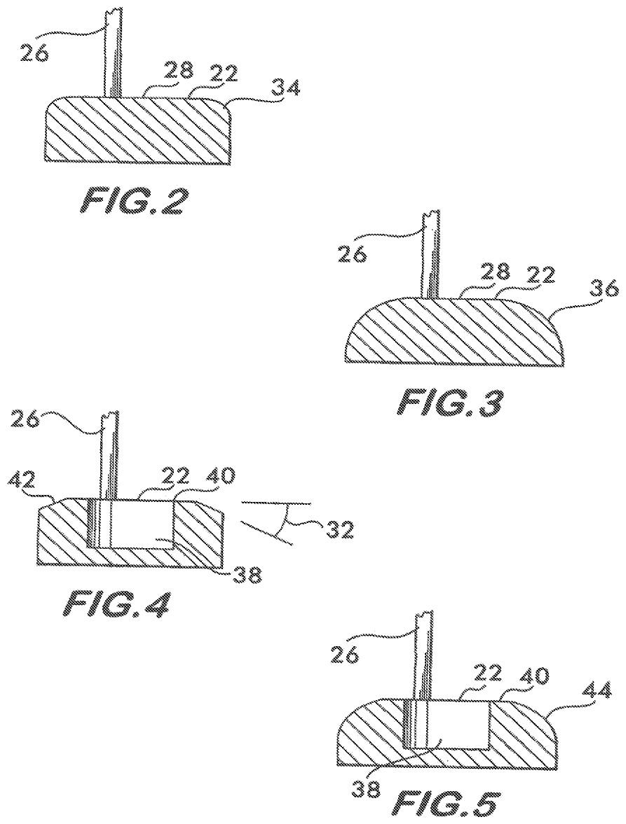

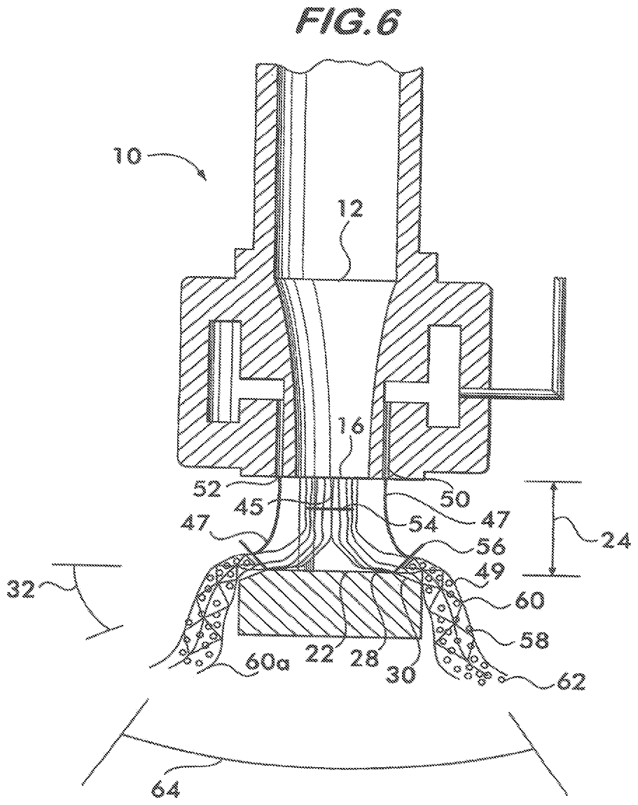

Deflector surface 22 may have other shapes, such as the curved upper edge 34 shown in FIG. 2 and the curved edge 36 shown in FIG. 3. As shown in FIGS. 4 and 5, the deflector surface 22 may also include a closed end resonance tube 38 surrounded by a flat portion 40 and a swept back, angled portion 42 (FIG. 4) or a curved portion 44 (FIG. 5). The diameter and depth of the resonance cavity may be approximately equal to the diameter of outlet 16.

With reference again to FIG. 1, an annular chamber 46 surrounds nozzle 12. Chamber 46 is in fluid communication with a pressurized supply of liquid decontamination agent 48 that provides the decontamination agent to the chamber at a predetermined pressure and flow rate. A plurality of ducts 50, separate from the nozzle 12, extend from the chamber 46. Each duct has an exit orifice 52 positioned adjacent to nozzle outlet 16. The exit orifices have a diameter between about 1/32 and ⅛ inches. Preferred distances between the nozzle outlet 16 and the exit orifices 52 range from about 1/64 inch to about ⅛ inch as measured along a radius line from the edge of the nozzle outlet to the closest edge of the exit orifice. The liquid decontamination agent include sterilizing agents as well as disinfecting agents and may be tailored to a specific target contaminant, for example bacterial spores, mycobacterial, small non-enveloped viruses, fungi, vegetative bacteria as well as lipid or medium size viruses. Candidate decontamination agents include, for example, hydrogen peroxide, formaldehyde, ethylene oxide, chlorine dioxide, peracetic acid, peroxides, alcohols, phenolics, halogens including bromine, chlorine, iodine, glutaraldehyde OPA, cidex OPA, ammonium compounds, aqueous silver solutions, aqueous copper solutions, glycols, sodium hypochloride and combinations thereof. The decontamination agent flows from the pressurized supply 48 into the chamber 46 and through the ducts 50, exiting from each orifice 52 where it is atomized by the gas flow from the pressurized gas supply 18 that flows through the nozzle 12 and exits through the nozzle outlet 16 as described in detail below. Emitter 10, when configured for use in a biological decontamination system, is designed to operate with a preferred gas pressure between about 29 psia to about 60 psia at the nozzle inlet 14 and a preferred liquid decontamination agent pressure between about 1 psig and about 50 psig in chamber 46.

Operation of the emitter 10 is described with reference to FIG. 6 which is a drawing based upon Schlieren photographic analysis of an operating emitter. Gas 45 from the pressurized supply 18 exits the nozzle outlet 16 at about Mach 1.5 and impinges on the deflector surface 22. Simultaneously, liquid decontamination agent 47 from the pressurized supply 48 is discharged from exit orifices 52. Interaction between the gas 45 and the deflector surface 22 establishes a first shock front 54 between the nozzle outlet 16 and the deflector surface 22. A shock front is a region of flow transition from supersonic to subsonic velocity. Liquid decontamination agent 47 exiting the orifices 52 does not enter the region of the first shock front 54 in this flow regime.

A second shock front 56 forms proximate to the deflector surface at the border between the flat surface portion 28 and the angled surface portion 30. Liquid decontamination agent 47 discharged from the orifices 52 is entrained with the gas jet 45 proximate to the second shock front 56 forming a decontamination agent liquid-gas stream 60. One method of entrainment is to use the pressure differential between the pressure in the gas flow jet and the ambient. Shock diamonds 58 form in a region along the angled portion 30, the shock diamonds being confined within the decontamination agent liquid-gas stream 60, which projects outwardly and downwardly from the emitter. The shock diamonds are also transition regions between super and subsonic flow velocity and are the result of the gas flow being over-expanded as it exits the nozzle. Over-expanded flow describes a flow regime wherein the external pressure (i.e., the ambient atmospheric pressure in this case) is higher than the gas exit pressure at the nozzle. This produces oblique shock waves which reflect from the free jet boundary 49 marking the limit between the decontamination agent liquid-gas stream 60 and the ambient atmosphere. The oblique shock waves are reflected toward one another to create the shock diamonds.

Significant shear forces are produced in the decontamination agent liquid-gas stream 60, which ideally does not separate from the deflector surface, although the emitter is still effective if separation occurs as shown at 60a. The decontamination agent 47, entrained proximate to the second shock front 56, is subjected to these shear forces which are the primary mechanism for atomization. The decontamination agent 47 also encounters the shock diamonds 58, which are a secondary source of atomization.

Thus, the emitter 10 operates with multiple mechanisms of atomization which produce particles 62 of liquid decontamination agent less than 20 micro meters in diameter, the majority of the particles being measured at less than 5 micro meters. The smaller droplets are buoyant in air. This characteristic provides for a homogeneous distribution over the target volume for greater decontamination efficacy. Furthermore, the particles maintain significant momentum, allowing the decontamination liquid-gas stream 60 to overcome any errant vortices or air currents which might otherwise prevent full blanketing of the volume to be decontaminated. Measurements show the liquid-gas stream having a velocity of 1,200 ft/min 18 inches from the emitter, and a velocity of 700 ft/min 8 feet from the emitter. The sweep back angle 32 of the angled portion 30 of the deflector surface 22 provides significant control over the included angle 64 of the liquid-gas stream 60. Included angles of about 120° are achievable. Additional control over the dispersion pattern of the flow is accomplished by adjusting the gap 24 between the nozzle outlet 16 and the deflector surface.

During emitter operation it is further observed that ambient air in the target volume to be decontaminated is drawn into the gas stream exiting the nozzle, and is entrained in the liquid-gas stream 60 at a volumetric ratio as high as 40:1. This phenomenon increases the efficiency of distribution of the decontamination agent due to the high volume of air exchange within the target volume.

The emitter causes a temperature drop due to the atomization of the liquid decontamination agent into the extremely small particle sizes described above. The cooler temperatures allow lower concentrations of the liquid decontamination agent to be effective. The emitter is unlike resonance tubes in that it does not produce significant acoustic energy. Jet noise (the sound generated by air moving over an object) is the only acoustic output from the emitter. The emitter's jet noise has no significant frequency components higher than about 6 kHz (half the operating frequency of well-known types of resonance tubes) and does not contribute significantly to decontamination agent atomization.

Furthermore, the flow from the emitter is stable and does not separate from the deflector surface (or experiences delayed separation as shown at 60a) unlike the flow from resonance tubes, which is unstable and separates from the deflector surface, thus leading to inefficient atomization or even loss of atomization.

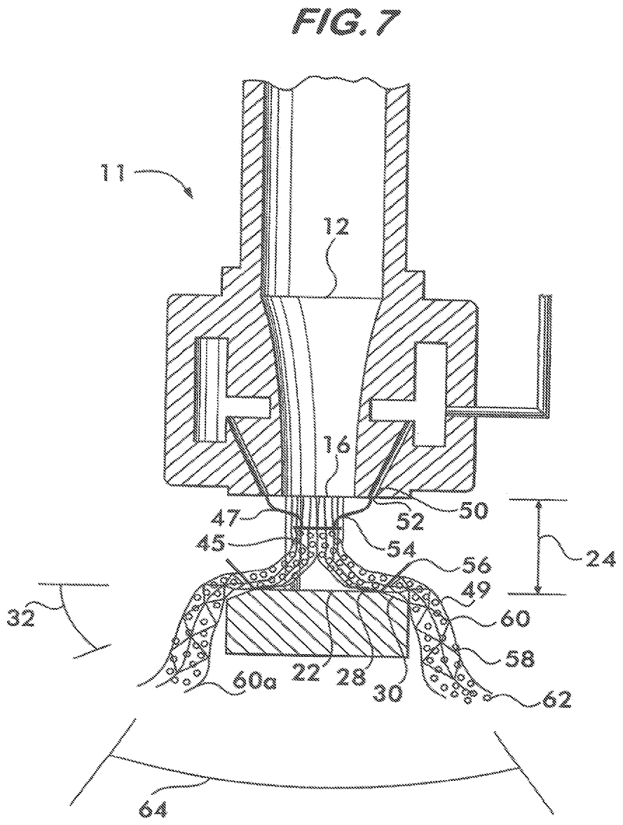

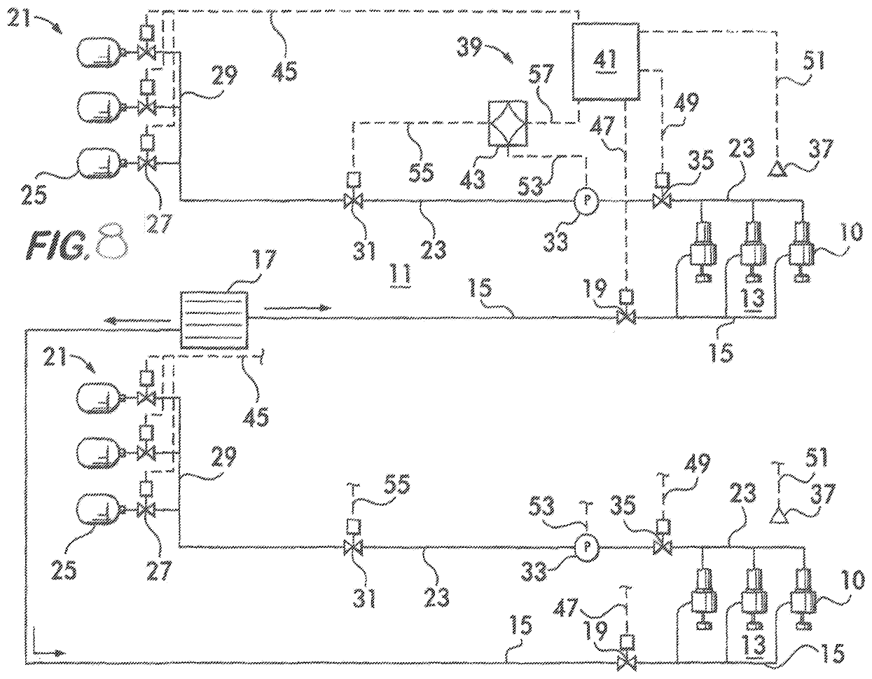

Another emitter embodiment 11 is shown in FIG. 7. Emitter 11 has ducts 50 that are angularly oriented toward the nozzle 12. The ducts are angularly oriented to direct the decontamination agent 47 toward the gas 45 so as to entrain the liquid in the gas proximate to the first shock front 54. It is believed that this arrangement will add yet another region of atomization in the creation of the decontamination liquid-gas stream 60 projected from the emitter 11.

Emitters according to the invention operated so as to produce an over-expanded gas jet with multiple shock fronts and shock diamonds achieve multiple stages of atomization and are expected to achieve increased decontamination effectiveness over the prior art when operated with a biological decontamination system as described below.

FIG. 8 illustrates, in schematic form, an example biological decontamination system 11 according to the invention. System 11 includes a plurality of high velocity low pressure emitters 10, described in detail above. Emitters 10 are arranged in the target area to be decontaminated. There is no limit to where system 11 can be effectively employed, For example, it is foreseen that the system 11 could be permanently installed in a room such as a hospital patient room, waiting room or office, an operatory for surgery or dental procedures, a testing laboratory as well as a lavatory or a bathroom. Industrial uses also abound, the system 11 being potentially useful in a research laboratory, a factory, a mill, an office building, in particular a post office or a logistics distribution center, as well as in agricultural applications such as food processing plants as well as farm buildings including barns and silos. It is further foreseen that mobile applications of the system are also feasible, as the components described below may be mounted on a vehicle such as a truck or a trailer and may be driven or towed on site to decontaminate the cabin spaces of boats, ships and aircraft. For clarity, a generic example is described herein, it being understood that the description is applicable to any of the applications described above as well as others.

In system 11 the emitters 10 are connected via a piping network 15 to a source of pressurized decontamination agent 17. Candidate liquid decontamination agents include, for example, hydrogen peroxide, formaldehyde, ethylene oxide, chlorine dioxide, peracetic acid, peroxides, alcohols, phenolics, halogens including bromine, chlorine, iodine, glutaraldehyde OPA, cidex OPA, ammonium compounds, aqueous silver solutions, aqueous copper solutions, glycols, sodium hypochloride and combinations thereof. A liquid control valve 19 controls the flow of decontamination agent from the source 17 to the emitters 10. The emitters are also in fluid communication with a source of pressurized gas 21 through a gas conduit network 23. The pressurized gas, for example air, nitrogen, oxygen, argon-nitrogen mixtures, argon-carbon dioxide mixtures or may also comprise a biological decontamination agent such as ozone, halogens, halocarbons and ethylene oxide, is maintained in banks of high-pressure cylinders 25. Cylinders 25 may be pressurized up to 2,500 psig. For large systems which require large volumes of gas, one or more lower pressure tanks (about 350 psig) having volumes on the order of 30,000 gallons may be used.

Valves 27 of cylinders 25 are preferably maintained in an open state in communication with a high pressure manifold 29. Gas flow rate and pressure from the manifold to the gas conduit 23 are controlled by a high pressure gas control valve 31. Pressure in the conduit 23 downstream of the high pressure control valve 31 is monitored by a pressure transducer 33. Flow of gas to the emitters 10 in the contaminated zone 13 is further controlled by a low pressure valve 35 downstream of the pressure transducer. The contaminated zone 13 may be monitored by one or more devices 37. These devices may comprise cameras, oxygen sensors, temperature sensors, relative humidity sensors, relative saturation sensors, decontamination agent concentration sensors as well as other sensors to permit remote monitoring of the contaminated zone. In an example embodiment the emitters 10 could be permanently mounted in the ceiling or walls of a room or building where the rest of the apparatus of system 11 also resides. In another example, the emitters 10 could be at the ends of flexible hoses (23) which lead back to a mobile unit mounted on a truck or trailer on which the rest of the system apparatus is carried. In this embodiment the emitters 10 are then temporarily positioned in the contaminated zone 13, which could be an aircraft cabin, a mail sorting room in a post office, a meat processing plant, or any space which requires decontamination.

The system components thus described are coordinated and controlled by a control system 39, which comprises a microprocessor 41 having a control panel display (not shown), resident software, and a programmable logic controller 43. In an example embodiment, the control system communicates with the system components to receive information and issue control commands as follows.

Each cylinder 25 is monitored as to its pressure status by a supervisory loop 45 that communicates with the microprocessor 41, which provides a visual indication of the cylinder pressure and may also indicate valve status (open or closed). Liquid control valve 19 is also in communication with microprocessor 41 via a communication line 47, which allows the valve 19 to be monitored and controlled (opened and closed) by the control system. Similarly, gas control valve 35 communicates with the control system via a communication line 49, and the sensor devices 37 also communicate with the control system via communication lines 51. The pressure transducer 35 provides its signals to the programmable logic controller 43 over communication line 53. The programmable logic controller is also in communication with the high pressure gas valve 31 over communication line 55, and with the microprocessor 41 over communication line 57.

In operation, sensors 37 may be used to display real time information from the contaminated zone 13 and provide video to operators as well as other sensor data (oxygen level, temperature, relative humidity, relative saturation, decontamination agent concentration) via signals to the microprocessor 41 over communication line 51. Upon commands from the operator the microprocessor actuates the logic controller 43. Note that controller 43 may be a separate controller or an integral part of the high pressure control valve 31. The logic controller 43 receives a signal from the pressure transducer 33 via communication line 53 indicative of the pressure in the gas conduit 23. The logic controller 43 opens the high pressure gas valve 31 while the microprocessor 41 opens the gas control valve 35 and the liquid control valve 19 using respective communication lines 49 and 47. Gas from tanks 25 and liquid decontamination agent from source 17 are thus permitted to flow through gas conduit 23 and liquid piping network 15 respectively. Preferred liquid decontamination agent pressure for proper operation of the emitters 10 is between about 1 psig and about 50 psig as described above. The logic controller 43 operates valve 31 to maintain the correct gas pressure (between about 29 psia and about 60 psia) and flow rate to operate the emitters 10 within the parameters as described above. At the command of the operator, the microprocessor 41 closes the gas and liquid valves 35 and 19, and the logic controller 43 closes the high pressure control valve 31. The sensors 37 may be used to continue monitoring the now decontaminated zone 13, and, if necessary the above described sequence may be repeated. Manual methods of system operation are also provided. For example, mechanical pressure reducing valves may be used for the gas control valve 35 and the liquid control valve 19 instead of valves actively controlled in a feedback loop by the logic controller 43. In the manually operated system an operator manually opens the pressure reducing gas control valve 35 and the pressure reducing liquid control valve 19 to permit flow of gas and decontamination agent to the emitter 10, with pressure control of the gas and decontamination agent occurring mechanically within each valve by a spring actuated closure element.

Whether the system is controlled manually or under microprocessor control, it is expected that the quantity of decontamination agent introduced into a target volume, the length of time of the discharge, and the dwell time of the agent in the room will be dependent upon or selected based upon the choice of decontamination agent employed, whether the pressurized gas is effectively inert or itself a decontamination agent, the environmental conditions of the target volume (such as relative humidity), feedback from the sensor devices, the contaminants of concern, and the extent or degree of decontamination required. It is anticipated that this system will, by appropriate selection and control of the above variables, be able to achieve decontamination levels consistent with disinfection but up to and including sterilization.

Emitters and decontaminating systems and methods according to the invention are expected to have short cycle times, high efficacy, provide a homogeneous distribution of decontamination agent over the contaminated volume, operate easily with a high degree of automation, require minimal safety measures and be environmentally friendly. These characteristics are expected to improve decontamination capabilities and efforts and thereby safeguard people against biological pathogens.

What is claimed is:

1. An emitter for atomizing and discharging a decontamination agent entrained in a gas stream, said emitter being connectable in fluid communication with a pressurized source of said decontamination agent and a pressurized source of said gas, said emitter comprising:
a nozzle having an inlet and an outlet and an unobstructed bore therebetween, said outlet having a diameter, said inlet being connectable in fluid communication with said pressurized gas source;
a duct, separate from said nozzle and connectable in fluid communication with said pressurized source of said decontamination agent, said duct having an exit orifice separate from and positioned adjacent to said nozzle outlet; and
a deflector surface positioned facing said nozzle outlet, said deflector surface being positioned in spaced relation to said nozzle outlet and having a first surface portion comprising a flat surface oriented substantially perpendicularly to said nozzle and a second surface portion comprising an angled or a curved surface surrounding said flat surface, said flat surface having a wetted area defined by a minimum diameter approximately equal to said outlet diameter, said decontamination agent being dischargeable from said orifice, and said gas being dischargeable from said nozzle outlet, said decontamination agent being entrained with said gas and atomized forming a decontamination agent liquid-gas stream that is deflected by said wetted area of said deflector surface and flows away therefrom.

2. The emitter according to claim 1, wherein said decontamination agent is selected from the group consisting essentially of hydrogen peroxide, formaldehyde, ethylene oxide, chlorine dioxide, peracetic acid, peroxides, alcohols, phenolics, halogens including bromine, chlorine, iodine, glutaraldehyde OPA, cidex OPA, ammonium compounds, aqueous silver solutions, aqueous copper solutions, glycols, sodium hypochloride and combinations thereof.

3. The emitter according to claim 1, wherein said gas is selected from the group consisting essentially of air, nitrogen, oxygen, argon-nitrogen mixtures, argon-carbon dioxide mixtures or may also comprise a biological decontamination agent such as ozone, halogens, halocarbons and ethylene oxide and combinations thereof.

4. A method of operating an emitter discharging a decontamination agent, said emitter comprising:
a nozzle having an unobstructed bore positioned between an inlet connectable in fluid communication with a pressurized gas source and an outlet having a diameter;
a duct connectable in fluid communication with a pressurized source of decontamination agent, said duct having an exit orifice positioned adjacent to said outlet; a deflector surface positioned facing said outlet in spaced relation thereto, said deflector surface comprising a flat surface oriented substantially perpendicularly to said nozzle, said flat surface having a wetted area defined by a minimum diameter approximately equal to said outlet diameter; said method comprising:
discharging said decontamination agent from said orifice;
discharging said gas from said outlet, said gas reaching supersonic speed;
establishing a first shock front between said outlet and said deflector surface wherein said gas slows to subsonic speed and then impinges on said wetted area;
establishing a second shock front proximate to said deflector surface, said gas moving across said wetted area and increasing to supersonic speed between said first shock front and said second shock front, and decreasing in speed after passing through said second shock front;
entraining said decontamination agent in said gas at least one of said shock fronts to form a decontamination liquid-gas stream;
projecting said decontamination liquid-gas stream from said emitter.

5. A method according to claim 4, comprising establishing a plurality of shock diamonds in said decontamination liquid-gas stream from said emitter.

6. A method according to claim 4, comprising creating an over-expanded gas flow jet after said gas is discharged from said nozzle.

7. A method according to claim 4, comprising supplying gas to said inlet at a pressure between about 29 psia and about 60 psia.

8. A method according to claim 4, comprising supplying decontamination agent to said duct at a pressure between about 1 psig and about 50 psig.

9. A method according to claim 4, further comprising entraining said decontamination agent with said gas proximate to said second shock front.

10. A method according to claim 4, further comprising entraining said decontamination agent with said gas proximate to said first shock front.

11. A method according to claim 4, wherein said decontamination liquid-gas stream does not separate from said deflector surface.

12. A method according to claim 4, comprising creating no significant acoustic energy from said emitter other than jet noise.

13. A method according to claim 4, further comprising generating momentum in said gas flow jet.

14. A method according to claim 4, further comprising projecting said decontamination liquid-gas stream at a velocity of about 1,200 ft/min at a distance of about 18 inches from said emitter.

15. A method according to claim 4, further comprising projecting said decontamination liquid-gas stream at a velocity of about 700 ft/min at a distance of about 8 feet from said emitter.

16. A method according to claim 4, further comprising establishing flow pattern from said emitter having a predetermined included angle by providing an angled portion of said deflector surface.

17. A method according to claim 4, comprising drawing said decontamination agent into said gas flow jet using a pressure differential between the pressure in said gas flow jet and the ambient.

18. A method according to claim 4, comprising entraining said decontamination agent into said gas flow jet and atomizing said decontamination agent into drops less than 20 micro meters in diameter.

19. A biological decontamination system, said system comprising:
a source of pressurized gas;
a source of pressurized decontamination agent;
at least one emitter for atomizing and discharging said decontamination agent entrained in said gas;
a gas conduit providing fluid communication between said pressurized gas source and said emitter;
a piping network, separate from said gas conduit, said piping network providing fluid communication between said pressurized source of decontamination agent and said emitter;
a first valve in said gas conduit controlling pressure and flow rate of said gas to said emitter;
a second valve in said piping network controlling pressure and flow rate of said decontamination agent to said emitter;
a pressure transducer measuring pressure within said gas conduit;
a control system in communication with said first and second valves and said pressure transducer, said control system receiving signals from said pressure transducer and opening and closing said valves in response to a signal indicative of said pressure within said gas conduit; wherein
said emitter comprises:
a nozzle having an inlet and an outlet and an unobstructed bore therebetween, said inlet being connected in fluid communication with said first valve, said outlet having a diameter;
a duct, separate from said nozzle and connected in fluid communication with said second valve, said duct having an exit orifice separate from and positioned adjacent to said nozzle outlet;
a deflector surface positioned facing said nozzle outlet, said deflector surface being positioned in spaced relation to said nozzle outlet and having a first surface portion comprising a flat surface oriented substantially perpendicularly to said nozzle and a second surface portion comprising an angled or a curved surface surrounding said flat surface, said flat surface having a wetted area defined by a minimum diameter approximately equal to said outlet diameter.

20. The system according to claim 19, further comprising:
a plurality of compressed gas tanks comprising said source of pressurized gas; and
a high pressure manifold providing fluid communication between said compressed gas tanks and said first valve.

21. The system according to claim 20, further comprising:
a plurality of control valves, each one being associated with one of said compressed gas tanks; and
a supervisory loop in communication with said control system and said control valves for monitoring the status of said control valves.

22. The system according to claim 19, wherein said system is installed in a room.

23. The system according to claim 22, wherein said room comprises a hospital room, an operatory, a laboratory or a bathroom.

24. The system according to claim 19, wherein said system is installed in a vehicle.

25. The system according to claim 24, wherein said vehicle comprises an automobile, a truck, a trailer, a ship, a boat or an aircraft.

26. The system according to claim 19, wherein said system is installed in a building.

27. The system according to claim 26, wherein said building comprises a hospital, a research laboratory, an office building, a factory, a mill, a silo, a barn or a post office.

28. A method of operating a biological decontamination system, said system having an emitter comprising:
a nozzle having an unobstructed bore positioned between an inlet and an outlet, said nozzle inlet being connected in fluid communication with a pressurized gas source, said outlet having a diameter;
a duct, separate from said nozzle and connected in fluid communication with a pressurized source of decontamination agent, said duct having an exit orifice positioned adjacent to said nozzle outlet;
a deflector surface positioned facing said nozzle outlet in spaced relation thereto, said deflector surface comprising a flat surface oriented substantially perpendicularly to said nozzle, said flat surface having a wetted area defined by a minimum diameter approximately equal to said outlet diameter; said method comprising:
discharging said decontamination agent from said exit orifice;
discharging said gas from said nozzle outlet, said gas achieving supersonic speed;
establishing a first shock front between said outlet and said deflector surface wherein said gas slows to subsonic speed and then impinges on said wetted area;
establishing a second shock front proximate to said deflector surface, said gas moving across said wetted area and increasing to supersonic speed between said first shock front and said second shock front, and decreasing in speed after passing through said second shock front;
entraining said decontamination agent in said gas proximate to said second shock front to form a decontamination liquid-gas stream; and
projecting said decontamination liquid-gas stream from said emitter.

29. The method according to claim 28, wherein said system comprises:
a plurality of compressed gas tanks forming said source of pressurized gas;
a plurality of control valves, each one being associated with one of said compressed gas tanks;
a supervisory loop in communication with said control valves for monitoring the open and closed status of said control valves; and
said method comprising monitoring the status of said control valves and maintaining said control valves in an open configuration during operation of said system.

30. The method according to claim 28, comprising establishing a plurality of shock diamonds in said decontamination liquid-gas stream.

31. The method according to claim 28, comprising creating an over-expanded gas flow jet after exiting from said nozzle.

32. The method according to claim 28, comprising supplying gas to said inlet at a pressure between about 29 psia and about 60 psia.

33. The method according to claim 28, comprising supplying said decontamination agent to said duct at a pressure between about 1 psig and about 50 psig.

34. The method according to claim 28, further comprising entraining said decontamination agent with said gas proximate to said first shock front.

35. The method according to claim 28, comprising creating no significant acoustic energy from said emitter other than jet noise.

36. The method according to claim 28, further comprising generating momentum in said gas flow jet.

37. The method according to claim 28, further comprising projecting said decontamination liquid-gas stream at a velocity of about 1,200 ft/min at a distance of about 18 inches from said emitter.

38. The method according to claim 28, further comprising projecting said decontamination liquid-gas stream at a velocity of about 700 ft/min at a distance of about 8 feet from said emitter.

39. The method according to claim 28, comprising drawing said decontamination agent into said gas using a pressure differential between the pressure in said gas and the ambient.

40. The method according to claim 28, comprising entraining said decontamination agent into said gas and atomizing said decontamination agent into drops less than 20 micro meters in diameter.

41. A method of operating a biological decontamination system, said system having an emitter comprising:
a nozzle having an unobstructed bore positioned between an inlet and an outlet, said nozzle inlet being connected in fluid communication with a pressurized gas source, said outlet having a diameter;
a duct, separate from said nozzle and connected in fluid communication with a pressurized source of decontamination agent, said duct having an exit orifice positioned adjacent to said nozzle outlet;
a deflector surface positioned facing said nozzle outlet in spaced relation thereto, said deflector surface comprising a flat surface oriented substantially perpendicularly to said nozzle, said flat surface having a wetted area defined by a minimum diameter approximately equal to said outlet diameter;
said method comprising:
discharging said decontamination agent from said exit orifice;
discharging said gas from said nozzle outlet, said gas achieving supersonic speed;
establishing a first shock front between said outlet and said deflector surface wherein said gas slows to subsonic speed and then impinges on said wetted surface;
establishing a second shock front proximate to said deflector surface, said gas moving across said wetted area and increasing to supersonic speed between said first shock front and said second shock front, and decreasing in speed after passing through said second shock front;
entraining said decontamination agent in said gas at at least one of said shock fronts to form a decontamination liquid-gas stream; and
projecting said decontamination liquid-gas stream from said emitter.

42. A method according to claim 41, further comprising entraining said decontamination agent with said gas proximate to said second shock front.

43. A method according to claim 41, further comprising entraining said decontamination agent with said gas proximate to said first shock front.

44. A biological decontamination system, said system comprising:
a gaseous decontamination agent;
a liquid decontamination agent;
at least one emitter for atomizing and entraining said liquid decontamination agent in said gaseous decontamination agent and discharging said gaseous and liquid decontamination agents;
a gas conduit conducting said gaseous decontamination agent to said emitter;
a piping network conducting said liquid decontamination agent to said emitter;
a first valve in said gas conduit controlling pressure and flow rate of said gaseous decontamination agent to said emitter;
a second valve in said piping network controlling pressure and flow rate of said liquid decontamination agent to said emitter;
a pressure transducer measuring pressure within said gas conduit; wherein
said emitter comprises:
a nozzle having an inlet and an outlet and an unobstructed bore therebetween, said outlet having a diameter, said inlet connected with said gas conduit downstream of said first valve;
a duct connected in fluid communication with said piping network downstream of said second valve, said duct having an exit orifice positioned adjacent to said nozzle outlet;
a deflector surface positioned facing said nozzle outlet, said deflector surface being positioned in spaced relation to said nozzle outlet and having a first surface portion comprising a flat surface oriented substantially perpendicularly to a gas flow from said nozzle and a second surface portion comprising an angled or a curved surface surrounding said flat surface, said flat surface having a wetted area defined by a minimum diameter approximately equal to said outlet diameter; and
a control system in communication with said first and second valves and said pressure transduce, said control system receiving signals from said pressure transducer and opening and closing said valves in response thereto.

45. A system according to claim 44, further comprising:
a plurality of compressed gas tanks comprising a source of pressurized gaseous decontamination agent; and
a high pressure manifold providing fluid communication between said compressed gas tanks and said gas conduit upstream of said first valve.
